(19) United States
(12) Patent Application Publication
(10) Pub. No.: US 2025/0120606 A1
(43) Pub. Date: Apr. 17, 2025

(54) SPHYGMOMANOMETER AND METHOD FOR MEASURING BLOOD PRESSURE

(71) Applicant: OMRON HEALTHCARE Co., Ltd., Kyoto (JP)

(72) Inventor: Yukiya SAWANOI, Kyoto (JP)

(21) Appl. No.: 18/990,636

(22) Filed: Dec. 20, 2024

Related U.S. Application Data
(63) Continuation of application No. PCT/JP2023/018718, filed on May 19, 2023.

(30) Foreign Application Priority Data
Sep. 16, 2022 (JP) 2022-148239

(51) Int. Cl. A61B 5/025; A61B 5/022

(52) U.S. Cl. CPC A61B 5/025; A61B 5/02225; A61B 5/02233

(57) ABSTRACT

A sphygmomanometer configured to measure a blood pressure by compressing a part of a user to be measured with a cuff, the sphygmomanometer including a blood pressure measurement unit configured to, after a pressurization process of pressurizing a cuff pressure indicating an inner pressure of the cuff to a pressure greater than a specified pressure, measure the blood pressure of the user based on a pulse wave signal in a depressurization process of depressurizing the cuff pressure, a determination unit configured to determine whether or not an arrhythmia occurs in the user based on the pulse wave signal in the depressurization process, and a mode setting unit configured to set one of a first mode for executing the determining of the arrhythmia and a second mode for not executing the determining of the arrhythmia.

### CROSS-REFERENCE TO RELATED APPLICATIONS

This application is the U.S. national stage application filed pursuant to 35 U.S.C. 365(c) and 120 as a continuation of International Patent Application No. PCT/JP2023/018718, filed May 19, 2023, which application claims priority to Japanese Patent Application No. 2022-148239, filed Sep. 16, 2022, which applications are incorporated herein by reference in their entireties.

### TECHNICAL FIELD

The present disclosure relates to a sphygmomanometer and a method for measuring a blood pressure.

### BACKGROUND ART

A technique for detecting atrial fibrillation using a signal acquired in the process of measuring a blood pressure is known from the prior art. For example, a sphygmomanometer according to Patent Document 1 (JP 2020-192322 A) determines atrial fibrillation through data of an interval time between pulse signals of pressurization stage measurement data and data of an interval time between pulse signals of depressurization stage measurement data.

### CITATION LIST

### Patent Literature

Patent Document 1: JP 2020-192322 A

### SUMMARY OF INVENTION

### Technical Problem

As a method of determining arrhythmia using a sphygmomanometer based on the oscillometric method, a method of determining arrhythmia such as atrial fibrillation based on a pattern of pulse wave intervals acquired during a depressurization process of a cuff pressure is known. For example, a rising point or a maximum point of a pulse wave signal for each beat is detected as a feature point, and an interval between a current beat and a beat one beat before is calculated as a pulse wave interval. In order to accurately calculate the pulse wave interval, the amplitude of the acquired pulse wave signal is preferably great. Therefore, in order to increase the accuracy of the arrhythmia determination based on the pattern of the pulse wave intervals, it is necessary to increase the amplitude of the pulse wave signal to acquire a more accurate pulse wave interval.

In one aspect, an object of the present disclosure is to provide a sphygmomanometer and a method for measuring a blood pressure capable of accurately determining arrhythmia by acquiring pulse wave signals having great amplitudes in a depressurization process of the blood pressure measurement.

### Solution to Problem

An example of the present disclosure provides a sphygmomanometer configured to measure a blood pressure by compressing a part of a user to be measured with a cuff. A sphygmomanometer includes a blood pressure measurement unit configured to, after a pressurization process of pressurizing a cuff pressure indicating an inner pressure of a cuff to a pressure greater than a specified pressure, measure a blood pressure of a user based on a pulse wave signal in a depressurization process of depressurizing the cuff pressure, a determination unit configured to determine whether or not an arrhythmia occurs in the user based on the pulse wave signal during the depressurization process, and a mode setting unit configured to set one of a first mode for executing the determining of arrhythmia and a second mode for not executing the determining of arrhythmia. The blood pressure measurement unit sets a first depressurization speed of the cuff pressure when the first mode is set to be lower than a second depressurization speed of the cuff pressure when the second mode is set.

According to the above configuration, the arrhythmia can be accurately determined by acquiring the pulse wave signals having great amplitudes in the depressurization process of the blood pressure measurement.

In another example of the present disclosure, the blood pressure measurement unit sets the first depressurization speed based on the pulse wave amplitude indicating the amplitude of the pulse wave signal in the pressurization process.

According to the above configuration, a more appropriate depressurization speed can be set for the user.

In another example of the present disclosure, the sphygmomanometer further includes a storage unit configured to store a pulse wave signal in a pressurization process or a depressurization process when the blood pressure of the user is measured by the blood pressure measurement unit. The blood pressure measurement unit sets the first depressurization speed based on a pulse wave amplitude indicating an amplitude of the pulse wave signal obtained in the past and stored in the storage unit.

According to the above configuration, a more appropriate depressurization speed can be set for the user.

In another example of the present disclosure, the blood pressure measurement unit sets the first depressurization speed to be lower the smaller the pulse wave amplitude.

According to the above configuration, a highly accurate arrhythmia determination can be realized in an appropriate measurement time.

In another example of the present disclosure, the blood pressure measurement unit sets the first depressurization speed to be lower the longer a time until the cuff pressure reaches a predetermined pressure in the pressurization process.

According to the above configuration, a highly accurate arrhythmia determination can be realized in an appropriate measurement time.

In another example of the present disclosure, the blood pressure measurement unit estimates a size of the cuff based on a time until the cuff pressure reaches a predetermined pressure in the pressurization process, and sets the first depressurization speed to be lower the greater the estimated size of the cuff.

According to the above configuration, a highly accurate arrhythmia determination can be realized in an appropriate measurement time.

In another example of the present disclosure, the sphygmomanometer further includes a winding strength detection unit configured to detect a winding strength of the cuff with respect to the part of the user to be measured based on the cuff pressure in the pressurization process and a capacity change of the cuff. The blood pressure measurement unit sets the first depressurization speed to be lower the smaller the winding strength.

According to the above configuration, a highly accurate arrhythmia determination can be realized in an appropriate measurement time.

In another example of the present disclosure, the blood pressure measurement unit sets the second depressurization speed based on the estimated pulse pressure that is a difference between the estimated systolic blood pressure and the estimated diastolic blood pressure, and a predetermined pulse rate necessary for measuring the blood pressure of the user.

According to the above configuration, rapid blood pressure measurement can be realized for the user who does not desire the arrhythmia determination.

According to another example of the present disclosure, there is provided a method of measuring a blood pressure by a sphygmomanometer configured to measure a blood pressure by compressing a part of a user to be measured with a cuff. A blood pressure measurement method includes, after a pressurization process of pressurizing a cuff pressure indicating an inner pressure of a cuff to a pressure greater than a specified pressure, measuring a blood pressure of a user based on a pulse wave signal in a depressurization process of depressurizing the cuff pressure, determining whether or not an arrhythmia occurs in the user based on the pulse wave signal in the depressurization process, and setting one of a first mode for executing the determining of arrhythmia and a second mode for not executing the determining of arrhythmia. A first depressurization speed of the cuff pressure when the first mode is set is lower than a second depressurization speed of the cuff pressure when the second mode is set.

According to the above configuration, the arrhythmia can be accurately determined by acquiring the pulse wave signals having great amplitudes in the depressurization process of the blood pressure measurement.

### Advantageous Effects of Invention

According to the present disclosure, an arrhythmia can be accurately determined by acquiring pulse wave signals having great amplitudes in a depressurization process of the blood pressure measurement.

### BRIEF DESCRIPTION OF DRAWINGS

FIG. 1 is a diagram illustrating a sphygmomanometer according to an embodiment.

FIG. 2 is a block diagram illustrating an example of a hardware configuration of the sphygmomanometer according to a first embodiment.

FIG. 3 is a block diagram illustrating a functional configuration of the sphygmomanometer according to the first embodiment.

FIG. 4 is a diagram illustrating a relationship between a pulse wave amplitude and a depressurization speed in a pressurization process.

FIG. 5 is a diagram illustrating a relationship between a pulse wave amplitude obtained in the past and a depressurization speed.

FIG. 6 is a flowchart illustrating an example of a processing procedure of the sphygmomanometer according to the first embodiment.

FIG. 7 is a flowchart illustrating another example of the processing procedure of the sphygmomanometer according to the first embodiment.

FIG. 8 is a flowchart illustrating still another example of the processing procedure of the sphygmomanometer according to the first embodiment.

FIG. 9 is a diagram for explaining a relationship between an arrival time to a predetermined pressure and a depressurization speed.

FIG. 10 is a diagram for explaining a relationship among an arrival time until a predetermined pressure is reached, a depressurization speed, and a cuff size.

FIG. 11 is a flowchart illustrating an example of a processing procedure of a sphygmomanometer according to a second embodiment.

FIG. 12 is a flowchart illustrating another example of the processing procedure of the sphygmomanometer according to the second embodiment.

FIG. 13 is a block diagram illustrating a functional configuration of a sphygmomanometer according to a third embodiment.

FIG. 14 is a flowchart illustrating an example of a processing procedure of the sphygmomanometer according to the third embodiment.

### DESCRIPTION OF EMBODIMENTS

Embodiments of the present invention will be described below with reference to the drawings. In the following description, like components are given like numerals. Names and functions thereof are also the same. Thus, the detailed description of such components is not repeated.

### Application Example

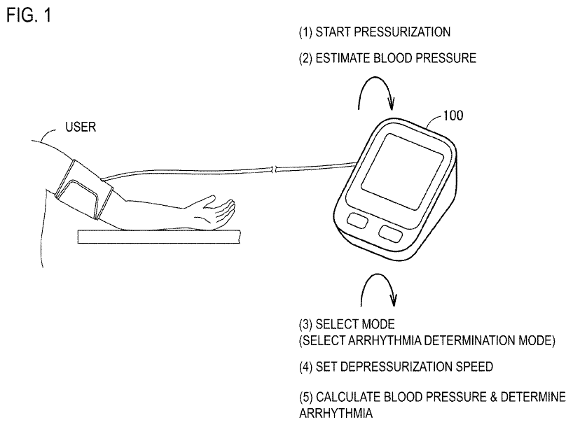

An application example of the present invention will be described with reference to FIG. 1. FIG. 1 is a diagram illustrating a sphygmomanometer 100 according to the present embodiment.

Referring to FIG. 1, the sphygmomanometer 100 is an upper-arm sphygmomanometer that measures a blood pressure by compressing a part of the user to be measured (i.e., the subject) with a cuff. The sphygmomanometer 100 executes blood pressure measurement through the oscillometric method. The sphygmomanometer 100 includes a main body and a cuff (arm band) as main components. The sphygmomanometer 100 may be a wrist-type sphygmomanometer in which a main body and a cuff (arm band) are integrated. Hereinafter, the processing contents will be described with reference to FIG. 1.

In FIG. 1, a scene in which a user measures his/her blood pressure using the sphygmomanometer 100 is assumed. The sphygmomanometer 100 measures the blood pressure of the user through a depressurization measurement method of measuring the blood pressure in a depressurization process of the cuff pressure indicating the inner pressure of the cuff worn on a part of the user to be measured (e.g., an arm).

The sphygmomanometer 100 starts pressurization of the cuff in response to a blood pressure measurement instruction of the user (corresponding to (1) in FIG. 1).

In the pressurization process of the cuff pressure, the sphygmomanometer 100 estimates the systolic blood pressure (maximum blood pressure) and the diastolic blood pressure (minimum blood pressure) based on the detected pulse wave signal (corresponding to (2) in FIG. 1). The systolic blood pressure and the diastolic blood pressure are estimated by a known method. For example, the sphygmomanometer 100 estimates the systolic blood pressure and the diastolic blood pressure from a pulse wave envelope indicating the pattern of the amplitude change of the pulse wave signal that changes in the process of pressurizing the cuff pressure. Hereinafter, the estimated systolic blood pressure is also referred to as “estimated systolic blood pressure”, and the estimated diastolic blood pressure is also referred to as “estimated diastolic blood pressure”.

The sphygmomanometer 100 selects either a normal measuring mode (hereinafter, also simply referred to as a “normal mode”) or an arrhythmia determination mode (corresponding to (3) in FIG. 1). The arrhythmia determination mode is a mode in which the arrhythmia determination is executed together with the blood pressure measurement based on the pulse wave signal in the depressurization process of the cuff pressure subsequent to the pressurization process. The normal mode is a mode in which only the blood pressure measurement is executed based on the pulse wave signal in the depressurization process and the arrhythmia determination is not executed. Here, it is assumed that the arrhythmia determination mode has been selected.

The sphygmomanometer 100 sets the depressurization speed of the cuff pressure in the depressurization process at the time of the arrhythmia determination mode (corresponding to (4) in FIG. 1). Here, at the time of the normal mode, the depressurization speed is set such that a predetermined pulse rate is obtained between the estimated pulse pressures that is the difference between the estimated systolic blood pressure and the estimated diastolic blood pressure. The depressurization speed in the arrhythmia determination mode is set to be lower than the depressurization speed in the normal mode. It is known that reducing the depressurization speed increases the amplitude of the pulse wave signal (hereinafter, “pulse wave amplitude”) obtained in the depressurization process. Therefore, the pulse wave amplitude obtained in the depressurization process at the time of the arrhythmia determination mode is greater than the pulse wave amplitude obtained in the depressurization process at the time of the normal mode.

The sphygmomanometer 100 calculates the blood pressure value of the user and determines the presence or absence of occurrence of arrhythmia based on the pulse wave signal obtained in the depressurization process (corresponding to (5) in FIG. 1). In this case, the sphygmomanometer 100 displays the blood pressure value of the user and the determination result of the arrhythmia on the display.

According to the above application example, since the pulse wave amplitude obtained in the arrhythmia determination mode is greater than the pulse wave amplitude obtained in the normal mode, a more accurate pulse wave interval pattern can be obtained in the arrhythmia determination mode. Therefore, the sphygmomanometer 100 can execute a more accurate arrhythmia determination.

Note that the sphygmomanometer 100 creates a pulse wave envelope based on the amplitude of the acquired pulse wave signal and the cuff pressure, and calculates the blood pressure based on the pulse wave envelope. The sphygmomanometer 100 performs processing such as correction and smoothing of the pulse wave envelope so that the blood pressure can be calculated even when the acquired pulse wave signal is small and cannot be recognized as a pulse wave. Therefore, at the time of the normal mode in which the depressurization speed is high, the sphygmomanometer 100 can calculate the blood pressure in a short measurement time. Thus, the blood pressure measurement is quickly performed for the user who does not desire the arrhythmia determination.

As described above, in the sphygmomanometer 100 according to the present embodiment, the arrhythmia can be accurately determined by acquiring the pulse wave signals having great amplitudes during the depressurization process of the blood pressure measurement.

### Configuration Example

### First Embodiment

### (Hardware Configuration)

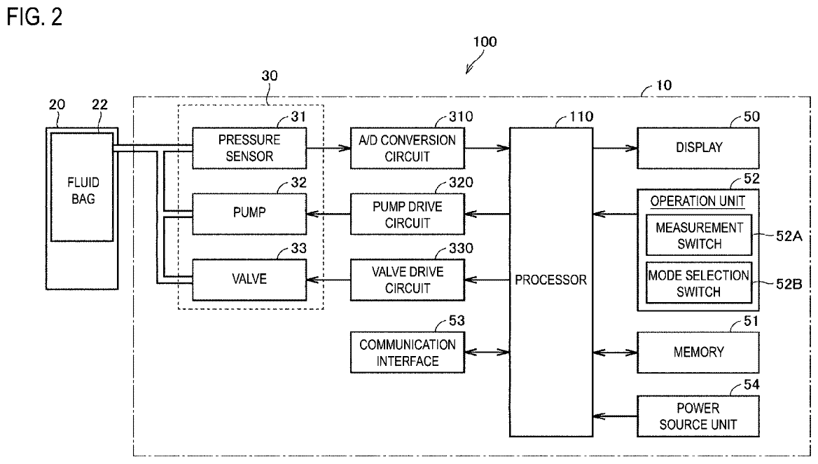

FIG. 2 is a block diagram illustrating an example of a hardware configuration of the sphygmomanometer 100 according to a first embodiment. Referring to FIG. 2, the sphygmomanometer 100 includes a main body 10 and a cuff 20 as main components. The cuff 20 interiorly includes a fluid bag 22. The main body 10 includes a processor 110, an air-system component 30 for blood pressure measurement, an A/D conversion circuit 310, a pump drive circuit 320, a valve drive circuit 330, a display 50, a memory 51, an operation unit 52, a communication interface 53, and a power source unit 54.

The processor 110 may typically be an arithmetic processing unit such as a Central Processing Unit (CPU) or a Multi Processing Unit (MPU). The processor 110 reads and executes the program stored in the memory 51 to implement each process (step) of the sphygmomanometer 100 described later. For example, the processor 110 performs control to drive the pump 32 and the valve 33 in response to an operation signal from the operation unit 52. In addition, the processor 110 calculates a blood pressure value using an algorithm for blood pressure calculation by an oscillometric method and displays the blood pressure value on the display 50.

The memory 51 is achieved by a Random Access Memory (RAM), a Read-Only Memory (ROM), a flash memory, or the like. The memory 51 stores a program for controlling the sphygmomanometer 100, data used for controlling the sphygmomanometer 100, setting data for setting various functions of the sphygmomanometer 100, data of a measurement result of a blood pressure value, a pulse rate, a pulse wave interval, and the like. Furthermore, the memory 51 is used as working memory and the like for when executing a program.

The air-system component 30 supplies or discharges air to or from the fluid bag 22 interiorly contained in the cuff 20 through an air line. The air-system component 30 includes a pressure sensor 31 for detecting a pressure in the fluid bag 22, and a pump 32 and a valve 33 serving as an expanding/contracting mechanism section for expanding/contracting the fluid bag 22.

The pressure sensor 31 detects a pressure (cuff pressure) in the fluid bag 22 and outputs a signal (cuff pressure signal) corresponding to the detected pressure to the A/D conversion circuit 310. The pressure sensor 31 is, for example, a piezo-resistive pressure sensor connected to the pump 32, the valve 33, and the fluid bag 22 interiorly contained in the cuff 20 via the air line. The pump 32 supplies air as a fluid to the fluid bag 22 through an air line in order to pressurize the cuff pressure. The valve 33 is opened and closed to control the cuff pressure by discharging air in the fluid bag 22 through the air line or enclosing air in the fluid bag 22.

The A/D conversion circuit 310 converts an output value of the pressure sensor 31 (e.g., a voltage value corresponding to a change in electric resistance due to a piezo-resistive effect) from an analog signal to a digital signal and outputs the converted signal to the processor 110. The processor 110 acquires a signal representing the cuff pressure according to the output value of the A/D conversion circuit 310. The pump drive circuit 320 controls driving of the pump 32 based on a control signal provided from the processor 110. The valve drive circuit 330 controls opening and closing of the valve 33 based on a control signal provided from the processor 110.

When the blood pressure is measured according to the oscillometric method, the following operation is generally performed. Specifically, a cuff is wound around a part of the subject to be measured (wrist, arm, etc.) in advance, and at the time of measurement, the pump 32 and the valve 33 are controlled to pressurize the cuff pressure to be higher than the estimated systolic blood pressure, and then gradually depressurize the cuff pressure. In the reducing pressure process, the cuff pressure is detected by the pressure sensor, and the variation of arterial volume generated in the artery at the part to be measured is determined to be a pulse wave signal. The systolic blood pressure and the diastolic blood pressure are calculated based on the change (mainly rise and fall) in the amplitude of the pulse wave signal accompanying the change in the cuff pressure at that time.

The operation unit 52 inputs an operation signal corresponding to an instruction by a user to the processor. The operation unit 52 includes a measurement switch 52A for receiving a blood pressure measurement instruction from the user, and a mode selection switch 52B for selecting a measuring mode.

When the measurement switch 52A is pressed, the part to be measured is temporarily compressed by the cuff 20, and the blood pressure measurement is executed through the oscillometric method. When the measurement switch 52A is pressed again during the blood pressure measurement, the blood pressure measurement is stopped.

Furthermore, when the mode selection switch 52B is pressed, the measuring mode is switched. For example, when the mode selection switch 52B is pressed in a case where the current measuring mode is set to the normal mode, the measuring mode is switched to the arrhythmia determination mode.

The display 50 displays various kinds of information including a blood pressure measurement result and the like based on a control signal from the processor 110. The communication interface 53 exchanges various kinds of information with an external device. The power source unit 54 supplies power to the processor 110 and each piece of hardware.

### (Functional Configuration)

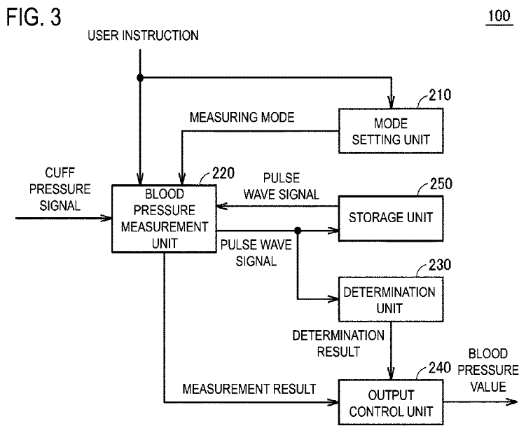

FIG. 3 is a block diagram illustrating a functional configuration of the sphygmomanometer 100 according to the first embodiment. Referring to FIG. 3, the sphygmomanometer 100 includes a mode setting unit 210, a blood pressure measurement unit 220, a determination unit 230, and an output control unit 240 as a main functional configuration. Each of these functions is realized, for example, by the processor 110 of the sphygmomanometer 100 executing a program stored in the memory 51. Note that some or all of these functions may be configured to be realized by hardware. The sphygmomanometer 100 further includes a storage unit 250. The storage unit 250 is realized by the memory 51.

The mode setting unit 210 sets one of an arrhythmia determination mode in which determination of presence or absence of arrhythmia of the user is executed and a normal mode in which determination of arrhythmia is not executed. Typically, the mode setting unit 210 sets either the arrhythmia determination mode or the normal mode in response to a mode selection instruction from the user via the operation unit 52 (e.g., the mode selection switch 52B).

Note that the mode setting unit 210 may be configured to automatically set either mode according to a schedule defined in advance. For example, when the blood pressure measurement is started (e.g., blood pressure measurement is started by pressing the measurement switch 52A) in the time zone H of the day, the arrhythmia determination mode is automatically set. On the other hand, when the blood pressure measurement is executed in a time zone other than the time zone H in the day, the normal mode is automatically set.

The blood pressure measurement unit 220 controls the cuff pressure in response to a measurement start instruction from the user via the operation unit 52 (e.g., the measurement switch 52A). Specifically, the blood pressure measurement unit 220 drives the pump 32 via the pump drive circuit 320 and performs a control for driving the valve 33 via the valve drive circuit 330. The valve 33 is opened and closed to discharge or enclose the air in the fluid bag 22 and control the cuff pressure.

The blood pressure measurement unit 220 receives the cuff pressure signal detected by the pressure sensor 31 and takes out a pulse wave signal representing the pulse wave of the part to be measured superimposed on the cuff pressure signal. That is, the blood pressure measurement unit 220 detects a pulse wave, which is a pressure component superimposed on the cuff pressure signal in synchronization with the pulsation of the heart of the user, from the cuff pressure signal.

The blood pressure measurement unit 220 calculates blood pressure information of the user based on the cuff pressure signal and the pulse wave signal superimposed on the cuff pressure signal. Specifically, the blood pressure measurement unit 220 measures the blood pressure of the user according to the oscillometric method. In the present embodiment, a depressurization measurement method is adopted in which after a pressurization process of pressurizing the cuff pressure to a pressure greater than a specified pressure (e.g., the estimated systolic blood pressure), the blood pressure of the user is measured based on a pulse wave signal in a depressurization process of depressurizing the cuff pressure. Typically, the blood pressure measurement unit 220 calculates a systolic blood pressure, a diastolic blood pressure, a pulse rate, and a pulse pressure. The storage unit 250 stores information obtained at the time of blood pressure measurement (e.g., pulse wave signal, systolic blood pressure, diastolic blood pressure, pulse rate, pulse pressure, etc.).

The blood pressure measurement unit 220 sets the depressurization speed of the cuff pressure in the depressurization process based on the measuring mode set by the mode setting unit 210. Specifically, the blood pressure measurement unit 220 makes the depressurization speed G1 of the cuff pressure when the arrhythmia determination mode is set lower than the depressurization speed G2 of the cuff pressure when the normal mode is set.

Typically, the blood pressure measurement unit 220 obtains the estimated systolic blood pressure and the estimated diastolic blood pressure based on the pulse wave signal in the pressurization process, and sets the depressurization speed G2 based on the estimated pulse pressure that is the difference between the estimated systolic blood pressure and the estimated diastolic blood pressure and a predetermined pulse rate (e.g., eight beats) necessary for measuring the blood pressure of the user. For example, the sphygmomanometer 100 sets the depressurization speed G2 such that the pulse rate generated between the estimated pulse pressures becomes greater than or equal to a predetermined pulse rate.

As a method of setting the depressurization speed G1 by the blood pressure measurement unit 220, several methods can be considered. In one aspect, the blood pressure measurement unit 220 sets the depressurization speed G1 to a speed (e.g., 4 mmHg/s) defined in advance. This speed (e.g., 4 mmHg/s) is a speed sufficiently lower than the depressurization speed G2 set by the above method.

In another aspect, the blood pressure measurement unit 220 sets the depressurization speed G1 based on the pulse wave amplitude in the pressurization process. The blood pressure measurement unit 220 sets the depressurization speed G1 to be lower the smaller the pulse wave amplitude.

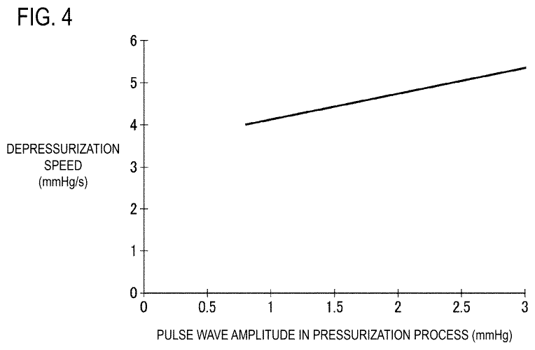

FIG. 4 is a diagram illustrating a relationship between a pulse wave amplitude and a depressurization speed in a pressurization process. In the graph of FIG. 4, the vertical axis represents the depressurization speed G1, and the horizontal axis represents the maximum pulse wave amplitude obtained in the pressurization process. It can be seen that the depressurization speed G1 is lower the smaller the maximum pulse wave amplitude in the pressurization process. This is because a highly accurate arrhythmia determination is performed by obtaining the pulse wave amplitude having a certain magnitude or more in the depressurization process.

Specifically, when the pulse wave amplitude in the pressurization process is great, there is a positive correlation that the pulse wave amplitude in the depressurization process is also great. Therefore, when the pulse wave amplitude in the pressurization process is small, the depressurization speed is reduced so that the pulse wave amplitude obtained in the depressurization process becomes great. On the other hand, when the pulse wave amplitude in the pressurization process is sufficiently great, it is considered that a great pulse wave amplitude is obtained also in the depressurization process, so that the depressurization speed is increased. In this case, the measurement time is shortened. In this manner, the depressurization speed G1 is set to a depressurization speed necessary for obtaining a pulse wave amplitude of a certain magnitude or more. As a result, a more appropriate depressurization speed can be set for the user, and a highly accurate arrhythmia determination can be realized in an appropriate measurement time.

Referring again to FIG. 3, the blood pressure measurement unit 220 sets the depressurization speed G1 based on the amplitude (i.e., pulse wave amplitude) of the pulse wave signal in the past stored in the storage unit 250.

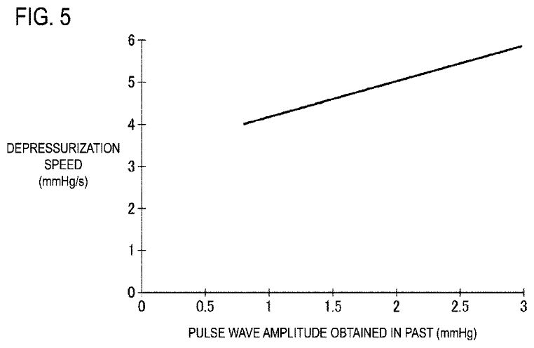

FIG. 5 is a diagram illustrating a relationship between a pulse wave amplitude obtained in the past and a depressurization speed. In the graph of FIG. 5, the vertical axis represents the depressurization speed G1, and the horizontal axis represents the pulse wave amplitude (hereinafter, also referred to as “pulse wave amplitude Am” for the sake of convenience) obtained in the pressurization process or the depressurization process at the time of the past blood pressure measurement of the user. It can be recognized that the depressurization speed G1 is lower the smaller the pulse wave amplitude Am. This is because a highly accurate arrhythmia determination is performed by obtaining the pulse wave amplitude having a certain magnitude or more in the depressurization process.

Specifically, when the pulse wave amplitude Am obtained in the past is small, it is estimated that the pulse wave amplitude in the depressurization process at the time of the current blood pressure measurement is also small. Therefore, when the pulse wave amplitude Am obtained in the past is small, the depressurization speed is reduced so that the pulse wave amplitude obtained in the current depressurization process becomes great. On the other hand, when the pulse wave amplitude Am obtained in the past is sufficiently great, it is considered that a great pulse wave amplitude can be obtained in the current depressurization process as well, and hence the depressurization speed is increased. In this case, the measurement time is shortened. In this manner, similarly to the case of FIG. 4, the depressurization speed G1 is set to a depressurization speed necessary for obtaining a pulse wave amplitude of a certain magnitude or more. Even in this case, a more appropriate depressurization speed can be set for the user, and a highly accurate arrhythmia determination can be realized in an appropriate measurement time.

The storage unit 250 stores the pulse wave amplitude in a past predetermined period. For example, the storage unit 250 stores a pulse wave signal in the pressurization process or the depressurization process when the blood pressure of the user is measured. As an example, the blood pressure measurement unit 220 calculates an average value of the maximum pulse wave amplitudes during the pressurization process at the time of a plurality of (e.g., three times) most recent blood pressure measurements based on the data stored in the storage unit 250, and sets the average value as the pulse wave amplitude Am. As another example, the blood pressure measurement unit 220 extracts data at the time of the past blood pressure measurement in the same time zone (e.g., morning, daytime, night, etc.) as the current blood pressure measurement, and sets the maximum pulse wave amplitude during the pressurization process at the time of the blood pressure measurement as the pulse wave amplitude Am. As still another example, the blood pressure measurement unit 220 sets, as the pulse wave amplitude Am, the maximum pulse wave amplitude during the pressurization process of a plurality of blood pressure measurements within a predetermined period (e.g., one day).

Referring to FIG. 3 again, when the arrhythmia determination mode is set, the determination unit 230 determines whether or not an arrhythmia of the user has occurred based on the pulse wave signal in the depressurization process. A known method is used for a method for determining arrhythmia. For example, the determination unit 230 determines the presence or absence of occurrence of arrhythmia based on the occurrence intervals (i.e., pulse wave interval) of the plurality of pulse waves acquired from the pulse wave signal.

The output control unit 240 displays the measurement result of the blood pressure measurement unit 220, the determination result of the determination unit 230, and the like on the display 50. Note that the output control unit 240 may transmit the measurement result and the determination result to an external device via the communication interface 53, or may be configured to output a voice via a speaker (not illustrated).

### (Processing Procedure)

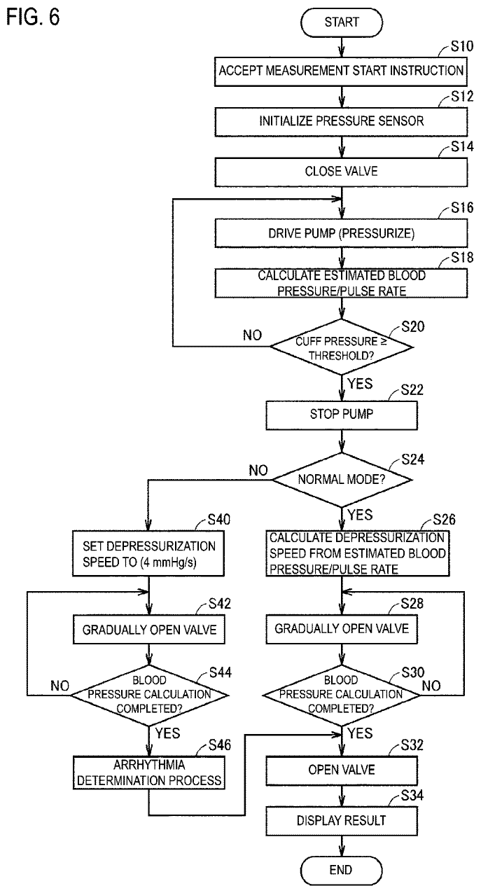

FIG. 6 is a flowchart illustrating an example of a processing procedure of the sphygmomanometer 100 according to the first embodiment. At the start of the process, it is assumed that the user is in a state of wearing the cuff 20 of the sphygmomanometer 100. In FIG. 6, it is assumed that the normal mode or the arrhythmia determination mode is set as the measuring mode.

Referring to FIG. 6, the processor 110 of the sphygmomanometer 100 accepts an instruction to start blood pressure measurement from the user via the measurement switch 52A of the operation unit 52 (step S10). The processor 110 initializes the pressure sensor 31 (step S12). Specifically, the processor 110 initializes the processing memory area, and performs 0 mmHg adjustment (setting the atmospheric pressure to 0 mmHg) of the pressure sensor 31 in a state where the pump 32 is turned off (stopped) and the valve 33 is opened.

Next, the processor 110 closes the valve 33 via the valve drive circuit 330 (step S14), and turns ON (activates) the pump 32 via the pump drive circuit 320 to start pressurization of the cuff 20 (fluid bag 22) (step S16). Typically, the processor 110 drives the pump 32 so that the pressurization speed of the cuff pressure is constant by controlling the pump drive circuit 320 for pressurization.

The processor 110 extracts the pulse wave signal from the cuff pressure signal detected by the pressure sensor 31, and calculates the estimated systolic blood pressure, the estimated diastolic blood pressure, and the pulse rate based on the pulse wave signal obtained in the pressurization process (step S18). Subsequently, the processor 110 determines whether or not the cuff pressure has reached higher than or equal to a threshold Th (step S20). Typically, the threshold Th is set to a value higher than the estimated systolic blood pressure by a fixed value (e.g., 40 mmHg).

When the cuff pressure is less than the threshold Th (NO in step S20), the processor 110 returns to step S16. When the cuff pressure is higher than or equal to the threshold Th (YES in step S20), the processor 110 stops the pump 32 (step S22), and determines whether or not the currently set measuring mode is the normal mode (step S24).

First, a case where the measuring mode is the normal mode (YES in step S24) will be described. The processor 110 calculates the depressurization speed G2 based on the estimated pulse pressure, which is the difference between the estimated systolic blood pressure and the estimated diastolic blood pressure, and the pulse rate (step S26). The processor 110 controls the valve 33 to be gradually opened according to the depressurization speed G2 (step S28). As a result, the process shifts from the pressurization process to the depressurization process, and the cuff pressures gradually reduces in accordance with the depressurization Speed G2.

In this depressurization process, the processor 110 extracts a pulse wave signal from the cuff pressure signal detected by the pressure sensor 31, attempts to calculate the systolic blood pressure and the diastolic blood pressure based on the pulse wave signal, and determines whether or not the blood pressure calculation is completed (step S30). When the blood pressure calculation cannot be completed yet due to lack of data (NO in step S30), the processor 110 returns to step S28. When the blood pressure calculation is completed (YES in step S30), the processor 110 performs a control to fully open the valve 33 (step S32) and rapidly exhaust the air in the cuff 20. The processor 110 displays the blood pressure value (measurement result) measured in step S30 on the display 50 (step S34).

Next, a case where the measuring mode is the arrhythmia determination mode (NO in step S24) will be described. The processor 110 sets the depressurization speed G1 to a predetermined speed (e.g., 4 mmHg/s) (step S40). The predetermined speed is assumed to be a speed sufficiently lower than the depressurization speed G2. The processor 110 controls the valve 33 to be gradually opened according to the depressurization speed G1 (step S42). As a result, the process shifts from the pressurization process to the depressurization process, and the cuff pressure is gradually depressurized according to the depressurization speed G1.

In the depressurization process, the processor 110 extracts the pulse wave signal from the cuff pressure signal, attempts to calculate the systolic blood pressure and the diastolic blood pressure based on the pulse wave signal, and determines whether the blood pressure calculation is completed (step S44). When the blood pressure calculation cannot be completed (NO in step S44), the processor 110 returns to step S42. When the blood pressure calculation is completed (YES in step S44), the processor 110 executes the arrhythmia determination process (step S46). Specifically, the processor 110 determines the presence or absence of occurrence of arrhythmia of the user based on the pulse wave interval acquired from the pulse wave signals in the depressurization process.

Subsequently, the processor 110 executes steps S32 and S34 described above. Note that in step S34, the blood pressure measurement result and the arrhythmia determination result are displayed as the measurement results.

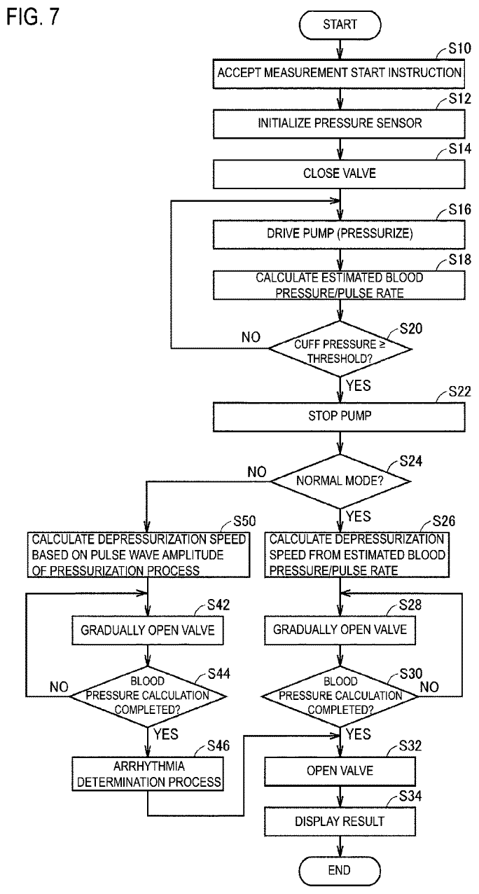

FIG. 7 is a flowchart illustrating another example of the processing procedure of the sphygmomanometer 100 according to the first embodiment. The flowchart of FIG. 7 corresponds to a flowchart in which step S40 in the flowchart of FIG. 6 is replaced with step S50. Therefore, the detailed description of the processes other than step S50 will not be repeated.

Referring to FIG. 7, in step S50, the processor 110 calculates the depressurization speed G1 based on the amplitude (i.e., pulse wave amplitude) of the pulse wave signal in the pressurization process. For example, the processor 110 calculates the depressurization speed G1 according to the graph of FIG. 4.

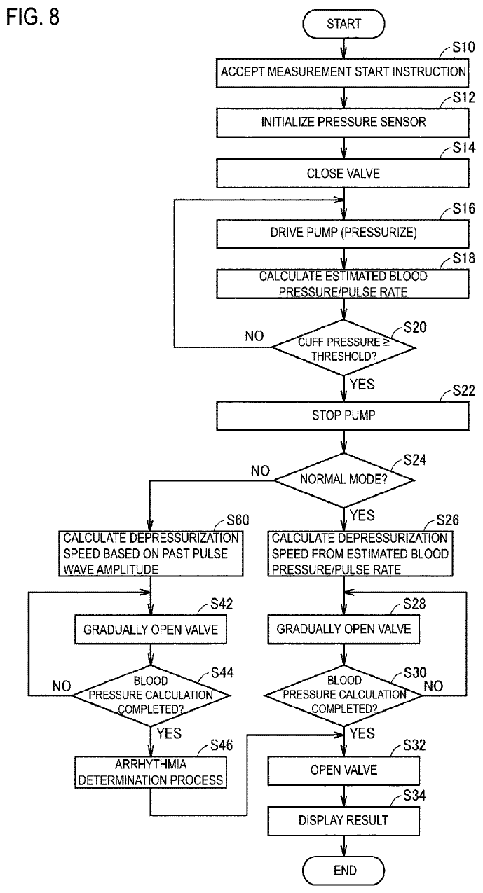

FIG. 8 is a flowchart illustrating still another example of the processing procedure of the sphygmomanometer 100 according to the first embodiment. The flowchart of FIG. 8 corresponds to a flowchart in which step S40 in the flowchart of FIG. 6 is replaced with step S60. Therefore, the detailed description of the processes other than step S60 will not be repeated.

Referring to FIG. 8, in step S60, the processor 110 calculates the depressurization speed G1 based on the pulse wave amplitude obtained in the past. For example, the processor 110 calculates the depressurization speed G1 according to the graph of FIG. 5.

### Second Embodiment

In a second embodiment, a method of setting the depressurization speed G1 focusing on the size of the cuff (e.g., the capacity of the fluid bag) will be described.

In order to accurately perform the blood pressure measurement, it is necessary to appropriately pressure-close the artery of the part to be measured by the cuff pressure. Therefore, the size of the cuff is determined according to the peripheral length of the part to be measured, and the size of the cuff becomes larger the longer the peripheral length of the part to be measured. Since the volume of the fluid bag increases as the size of the cuff increases, the ratio of the change in the volume of the fluid bag with respect to the change in the volume of the artery decreases. As a result, the change in the cuff pressure, i.e., the amplitude of the pulse wave signal (i.e., the pulse wave amplitude) becomes smaller. Hereinafter, a method of setting the depressurization speed G1 according to the second embodiment will be described.

### (Functional Configuration)

A functional configuration of the sphygmomanometer 100 according to the second embodiment will be described with reference to a block diagram of FIG. 3. The blood pressure measurement unit 220 according to the second embodiment further has the following functions in addition to the functions described with reference to FIG. 3.

In the pressurization process, the blood pressure measurement unit 220 drives the pump 32 to have a constant discharge flow rate per unit time to pressurize the cuff. The blood pressure measurement unit 220 measures an arrival time T until the cuff pressure reaches a predetermined pressure in the pressurization process based on the cuff pressure signal detected by the pressure sensor 31. The arrival time T changes according to the volume (size) of the cuff. For example, the blood pressure measurement unit 220 measures the arrival time T until the cuff pressure reaches “30 mmHg” from “0 mmHg”. The blood pressure measurement unit 220 sets the depressurization speed G1 to be lower the longer the arrival time T.

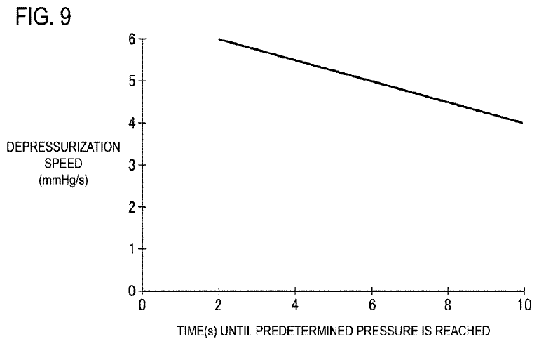

FIG. 9 is a diagram for explaining a relationship between an arrival time to a predetermined pressure and a depressurization speed. In the graph of FIG. 9, the vertical axis represents the depressurization speed G1, and the horizontal axis represents the arrival time T until the cuff pressure reaches “30 mmHg” from “0 mmHg”.

As illustrated in the graph of FIG. 9, it can be recognized that the depressurization speed G1 becomes lower the longer the arrival time T. This is because a highly accurate arrhythmia determination is performed by obtaining the pulse wave amplitude having a certain magnitude or more in the depressurization process.

Specifically, since the pump 32 is driven so as to have a constant discharge flow rate per unit time to pressurize the cuff pressure, longer arrival time T means greater capacity of the cuff (i.e., the size of the cuff). As described above, the pulse wave amplitude becomes smaller the larger the size of the cuff. Therefore, by setting the depressurization speed G1 to be lower the longer the arrival time T (i.e., the larger the size of the cuff), the pulse wave amplitudes obtained during the depressurization process can be increased. As a result, a highly accurate arrhythmia determination is realized.

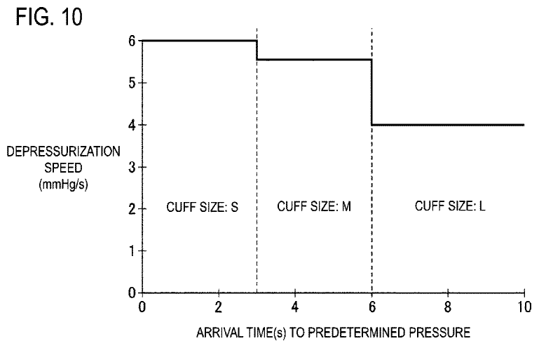

Note that the blood pressure measurement unit 220 may be configured to estimate the size of the cuff based on the arrival time T. FIG. 10 is a diagram for explaining a relationship among an arrival time until a predetermined pressure is reached, a depressurization speed, and a cuff size. In the graph of FIG. 10, the vertical axis represents the depressurization speed G1, and the horizontal axis represents the arrival time T.

The blood pressure measurement unit 220 estimates the cuff size to be “Small” when the arrival time T is less than three seconds, estimates the cuff size to be “Medium” when the arrival time T is longer than or equal to three seconds and less than six seconds, and estimates the cuff size to be “Large” when the arrival time Tis longer than or equal to six seconds. That is, the blood pressure measurement unit 220 estimates that the cuff size is larger the longer the arrival time T.

The blood pressure measurement unit 220 sets the depressurization speed G1 to 6 mmHg/s when the cuff size is estimated as “Small”, sets the depressurization speed G1 to 5.5 mmHg/s when the cuff size is estimated as “Medium”, and sets the depressurization speed G1 to 4 mmHg/s when the cuff size is estimated as “Large”. That is, the blood pressure measurement unit 220 sets the depressurization speed G1 to be lower the larger the cuff size.

### (Processing Procedure)

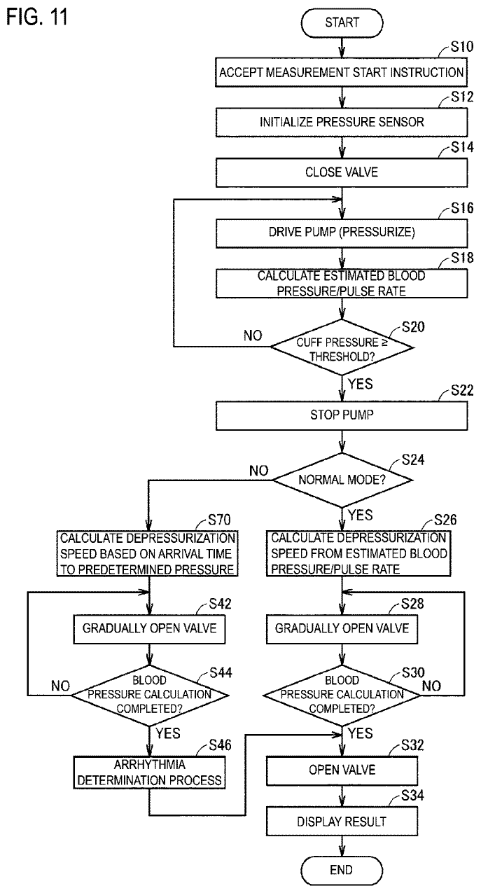

FIG. 11 is a flowchart illustrating an example of a processing procedure of the sphygmomanometer 100 according to the second embodiment. The flowchart of FIG. 11 corresponds to a flowchart in which step S40 in the flowchart of FIG. 6 is replaced with step S70. Therefore, the detailed description of the processes other than step S70 will not be repeated. However, in step S16, the processor 110 drives the pump 32 to have a constant discharge flow rate per unit time by controlling the pump drive circuit 320 for pressurization.

Referring to FIG. 11, in step S70, the processor 110 calculates the depressurization speed G1 based on the arrival time T until the cuff pressure reaches the predetermined pressure in the pressurization process. For example, the processor 110 calculates the depressurization speed G1 according to the graph of FIG. 9.

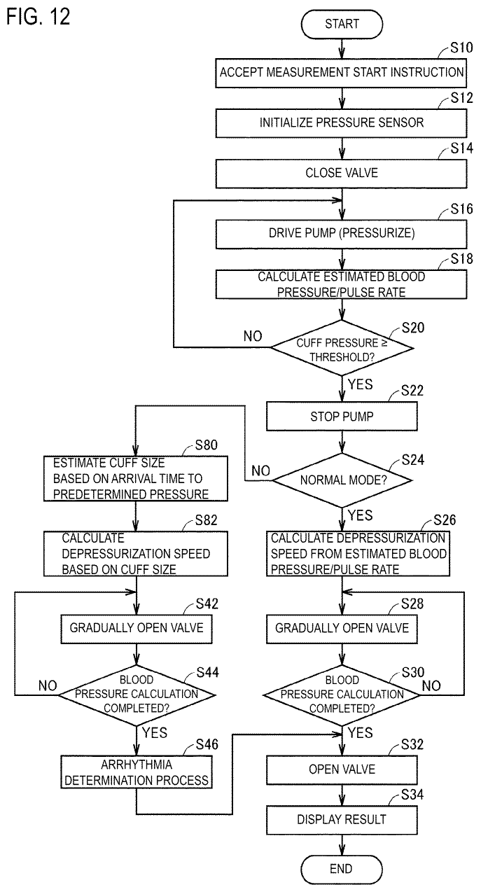

FIG. 12 is a flowchart illustrating another example of the processing procedure of the sphygmomanometer 100 according to the second embodiment. The flowchart of FIG. 12 corresponds to a flowchart in which step S40 in the flowchart of FIG. 6 is replaced with steps S80 and S82. Therefore, the detailed description of the processes other than steps S80 and S82 will not be repeated. However, in step S16, the processor 110 drives the pump 32 to have a constant discharge flow rate per unit time by controlling the pump drive circuit 320 for pressurization.

Referring to FIG. 12, the processor 110 estimates the cuff size based on the arrival time T (step S80), and calculates the depressurization speed G1 based on the estimated cuff size (step S82). Specifically, the processor 110 estimates the cuff size according to the graph of FIG. 10 and calculates the depressurization speed G1.

### Third Embodiment

In a third embodiment, a method of setting the depressurization speed G1 focusing on the tightening degree of the cuff will be described.

When the cuff is loosely wound, the fluid bag needs to be excessively inflated until the gap between the living body and the cuff is filled. Therefore, in a case where the cuff is loosely wound, the volume of the fluid bag becomes larger than that in a case where the cuff is tightly wound, and as a result, the amplitude of the obtained pulse wave signal becomes small. Hereinafter, a method of setting the depressurization speed G1 according to the third embodiment will be described.

### (Functional Configuration)

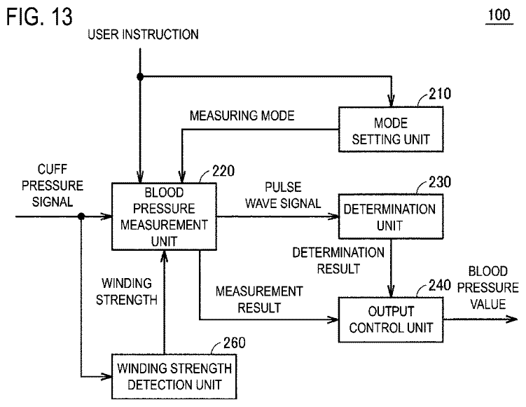

FIG. 13 is a block diagram illustrating a functional configuration of the sphygmomanometer 100 according to the third embodiment. Referring to FIG. 13, the sphygmomanometer 100 includes a mode setting unit 210, a blood pressure measurement unit 220, a determination unit 230, an output control unit 240, and a winding strength detection unit 260. Each of these functions is realized, for example, by the processor 110 of the sphygmomanometer 100 executing a program stored in the memory 51. The functions of the mode setting unit 210, the determination unit 230, and the output control unit 240 are similar to the functions described with reference to FIG. 3.

The winding strength detection unit 260 detects the winding strength of the cuff 20 with respect to the part to be measured based on the cuff pressure in the pressurization process and the change in the capacity of the cuff 20. The winding strength detection method is realized by a known method. Typically, the winding strength detection unit 260 has a similar function as the winding strength detection unit described in JP 5408142.

As an example, in the pressurization process of pressurizing the cuff by driving the pump 32 so as to have a constant discharge flow rate, the winding strength detection unit 260 compares the value of the capacity change of the cuff 20 detected with the change in the cuff pressure from the pressure P1 to the pressure P2 with the value of the capacity change of the cuff 20 detected with the change in the cuff pressure from the pressure P2 to the pressure P2 based on the pressure-capacity change relationship indicated by the capacity change of the cuff 20 detected with the change in the cuff pressure from the pressure P1 to the pressure P3 and the capacity change of the cuff 20 detected with the change in the cuff pressure from the pressure P2 to the pressure P3, and detects the winding strength of the cuff 20 from the comparison result.

The pressures P1, P2, and P3 are cuff pressures suitable for detecting the winding strength of the cuff determined in advance by an experiment or the like. Time when the cuff pressure reaches the pressures P1 to P3 is defined as time V1 to V3, respectively.

In this case, the winding strength detection unit 260 calculates a change ΔP12, which is a difference from the pressure P1 to the pressure P2, and a time ΔV12 indicating a time required for the cuff pressure to change by ΔP12 (i.e., the time V2−V1). The time ΔV12 is proportional to the change in fluid volume in the cuff as the pressure changes from the pressure P1 to the pressure P2. In this case, the winding strength detection unit 260 calculates a change ΔP23, which is a difference from the pressure P2 to the pressure P3, and a time ΔV23 indicating a time required for the cuff pressure to change by ΔP23 (i.e., the time V3−V2). The time ΔV23 is proportional to the change in fluid volume in the cuff as the pressure changes from the pressure P2 to the pressure P3.

The winding strength detection unit 260 calculates the pressure capacity change indexes ΔP12/ΔV12 and ΔP23/ΔV23, and compares the calculated values of both. The winding strength detection unit 260 detects the winding strength based on the comparison result.

In a case where the comparison result is (ΔP12/ΔV12)< (ΔP23/ΔV23), the winding strength detection unit 260 detects that the winding strength of the cuff 20 is “loose winding”. In a case where the comparison result is (ΔP12/ΔV12)>(ΔP23/ΔV23), the winding strength detection unit 260 detects that the winding strength of the cuff 20 is “tight winding”. In a case where the comparison result is (ΔP12/ΔV12)=(ΔP23/ΔV23), the winding strength detection unit 260 detects that the winding strength of the cuff 20 is “exact winding”.

“Loose winding” is a winding state in which the cuff 20 is loosely wound around the part to be measured and the pressurization on the part to be measured is lower than an appropriate level. “Exact winding” is a winding state in which the cuff 20 is appropriately wound around the part to be measured and the pressurization on the part to be measured is an appropriate level. “Tight winding” is a winding state in which the cuff 20 is loosely wound around the part to be measured and the pressurization on the part to be measured is higher than an appropriate level.

The blood pressure measurement unit 220 sets the depressurization speed G1 to be lower the smaller the winding strength detected by the winding strength detection unit 260. For example, for the sake of convenience, the depressurization speed G1 in the case where the winding strength is “loose winding” is set to “G1a”, the depressurization speed G1 in the case where the winding strength is “exact winding” is set to “G1b”, and the depressurization speed G1 in the case where the winding strength is “tight winding” is set to “G1c”. In this case, the depressurization speed G1a is the slowest, the depressurization speed G1c is the fastest, and the depressurization speed G1b is intermediate (i.e., G1a<G1b<G1c).

### (Processing Procedure)

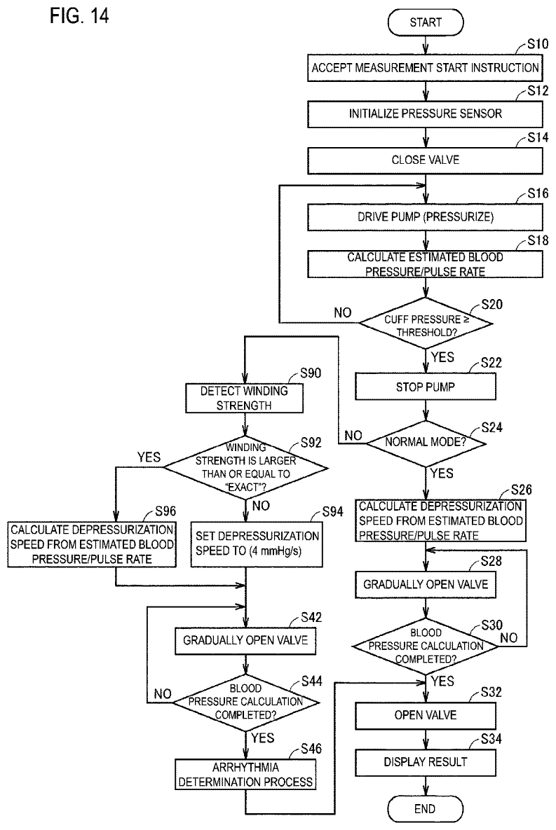

FIG. 14 is a flowchart illustrating an example of a processing procedure of the sphygmomanometer 100 according to the third embodiment. The flowchart of FIG. 14 corresponds to a flowchart in which step S40 in the flowchart of FIG. 6 is replaced with steps S90, S92, S94, and S96. Therefore, the detailed description of the processes other than steps S90, S92, S94, and S96 will not be repeated. However, in step S16, the processor 110 drives the pump 32 to have a constant discharge flow rate per unit time by controlling the pump drive circuit 320 for pressurization.

Referring to FIG. 14, the processor 110 detects the winding strength of the cuff 20 with respect to the part to be measured (step S90). The processor 110 determines whether or not the detected winding strength is greater than or equal to “exact winding” (i.e., “exact winding” or “tight winding”) (step S92). When the winding strength is greater than or equal to “exact winding” (YES in step S92), the processor 110 calculates the depressurization speed G1 based on the estimated pulse pressure, which is the difference between the estimated systolic blood pressure and the estimated diastolic blood pressure, and the pulse rate (step S96). When the winding strength is “loose winding” (NO in step S92), the processor 110 sets the depressurization speed G1 to a predetermined speed (e.g., 4 mmHg/s) (step S94).

In the flowchart of FIG. 14, the configuration in which the depressurization speed G1 is calculated based on the estimated pulse pressure and the pulse rate by the calculation method similar to that in the normal mode when the winding strength is greater than or equal to “exact winding” has been described, but the configuration is not limited to this configuration. For example, a configuration may be adopted in which the depressurization speed G1 is set to the depressurization speeds G1a to G1c described above in accordance with the winding strength (“loose winding”, “exact winding”, or “tight winding”).

### Other Embodiments

(1) In the above-described embodiment, a program may be provided that causes a computer to function and execute controls such as those described in the flowcharts described above. Such a program can also be provided as a program product recorded on a non-temporary computer-readable recording medium attached to a computer, such as a flexible disk, a compact disc read only memory (CD-ROM), a secondary storage device, a main storage device, and a memory card. Alternatively, a program may be provided by recording the program on a recording medium such as a hard disk built into a computer. The program may also be provided by download via a network.

(2) The configuration exemplified as the embodiment described above is an example of a configuration of the present invention, and the configuration can be combined with other known technology, and one part thereof may be omitted or modified within the scope not deviating from the gist of the present invention. Furthermore, the processes and configurations described in other embodiments may be employed as appropriate in the embodiments described above.

### [Supplementary Notes]

As described above, the present embodiments include the following disclosures.

### [Configuration 1]

A sphygmomanometer (100) configured to measure a blood pressure by compressing a part of a user to be measured with a cuff (20), the sphygmomanometer including a blood pressure measurement unit (220) configured to, after a pressurization process of pressurizing a cuff pressure indicating an inner pressure of the cuff to a pressure greater than a specified pressure, measure the blood pressure of the user based on a pulse wave signal in a depressurization process of depressurizing the cuff pressure, a determination unit (230) configured to determine whether or not an arrhythmia occurs in the user based on the pulse wave signal in the depressurization process, and a mode setting unit (210) configured to set one of a first mode for executing the determining of the arrhythmia and a second mode for not executing the determining of the arrhythmia, wherein the blood pressure measurement unit sets a first depressurization speed of the cuff pressure when the first mode is set to be lower than a second depressurization speed of the cuff pressure when the second mode is set.

### [Configuration 2]

The sphygmomanometer according to the configuration 1, wherein the blood pressure measurement unit sets the first depressurization speed based on a pulse wave amplitude indicating an amplitude of the pulse wave signal in the pressurization process.

### [Configuration 3]

The sphygmomanometer according to configuration 1, further including a storage unit (250) configured to store a pulse wave signal in the pressurization process or the depressurization process when the blood pressure of the user is measured by the blood pressure measurement unit, wherein the blood pressure measurement unit sets the first depressurization speed based on a pulse wave amplitude indicating an amplitude of the pulse wave signal in the past stored in the storage unit.

### [Configuration 4]

The sphygmomanometer according to configuration 2 or 3, wherein the blood pressure measurement unit sets the first depressurization speed to be lower the smaller the pulse wave amplitude.

### [Configuration 5]

The sphygmomanometer according to configuration 1, wherein the blood pressure measurement unit sets the first depressurization speed to be lower the longer a time until the cuff pressure reaches a predetermined pressure in the pressurization process.

### [Configuration 6]

The sphygmomanometer according to configuration 1, wherein the blood pressure measurement unit estimates a size of the cuff based on a time until the cuff pressure reaches a predetermined pressure in the pressurization process, and sets the first depressurization speed to be lower the larger the estimated size of the cuff.

### [Configuration 7]

The sphygmomanometer according to configuration 1, further including a winding strength detection unit (260) configured to detect a winding strength of the cuff with respect to the part of the user to be measured based on the cuff pressure in the pressurization process and a capacity change of the cuff, wherein the blood pressure measurement unit sets the first depressurization speed to be lower the smaller the winding strength.

### [Configuration 8]

The sphygmomanometer according to any one of configurations 1 to 7, wherein the blood pressure measurement unit sets the second depressurization speed based on an estimated pulse pressure that is a difference between an estimated systolic blood pressure and an estimated diastolic blood pressure, and a predetermined pulse rate necessary for measuring the blood pressure of the user.

### [Configuration 9]

A method for measuring a blood pressure by a sphygmomanometer (100) configured to measure a blood pressure by compressing a part of a user to be measured with a cuff (20), the method including measuring, after a pressurization process of pressurizing a cuff pressure indicating an inner pressure of the cuff to a pressure greater than a specified pressure, the blood pressure of the user based on a pulse wave signal in a depressurization process of depressurizing the cuff pressure, determining whether or not an arrhythmia occurs in the user based on the pulse wave signal in the depressurization process, and setting one of a first mode for executing the determining of the arrhythmia and a second mode for not executing the determining of the arrhythmia, wherein a first depressurization speed of the cuff pressure when the first mode is set is lower than a second depressurization speed of the cuff pressure when the second mode is set.

The embodiments disclosed herein are illustrative in all respects and are not intended as limitations. The scope of the present invention is indicated not by the descriptions above but by the claims and includes all meaning equivalent to the scope and changes within the scope.

### REFERENCE SIGNS LIST

10 Main body, 20 Cuff, 22 Fluid bag, 30 Air-system component, 31 Pressure sensor, 32 Pump, 33 Valve, 50 Display, 51 Memory, 52 Operation unit, 52A Measurement switch, 52B Mode selection switch, 53 Communication interface, 54 Power source unit, 100 Sphygmomanometer, 110 Processor, 210 Mode setting unit, 220 Blood pressure measurement unit, 230 Determination unit, 240 Output control unit, 250 Storage unit, 260 Winding strength detection unit, 310 A/D conversion circuit, 320 Pump drive circuit, 330 Valve drive circuit.

1. A sphygmomanometer configured to measure a blood pressure by compressing a part of a user to be measured with a cuff, the sphygmomanometer comprising:
a blood pressure measurement unit configured to, after a pressurization process of pressurizing a cuff pressure indicating an inner pressure of the cuff to a pressure greater than a specified pressure, measure the blood pressure of the user based on a pulse wave signal in a depressurization process of depressurizing the cuff pressure;
a determination unit configured to determine whether or not an arrhythmia occurs in the user based on the pulse wave signal in the depressurization process; and
a mode setting unit configured to set one of a first mode for executing the determining of the arrhythmia and a second mode for not executing the determining of the arrhythmia, wherein
the blood pressure measurement unit,
sets a first depressurization speed of the cuff pressure when the first mode is set to be lower than a second depressurization speed of the cuff pressure when the second mode is set; and
sets the first depressurization speed to be lower the smaller a pulse wave amplitude indicating an amplitude of the pulse wave signal in the pressurization process or sets the first depressurization speed to be lower the smaller a pulse wave amplitude indicating an amplitude of the pulse wave signal in a predetermined period in the past.

2. The sphygmomanometer according to claim 1, further comprising:
a storage unit configured to store a pulse wave signal in the pressurization process or the depressurization process when the blood pressure of the user is measured by the blood pressure measurement unit as the pulse wave signal in the predetermined period in the past.

3. A sphygmomanometer configured to measure a blood pressure by compressing a part of a user to be measured with a cuff, the sphygmomanometer comprising:
a blood pressure measurement unit configured to, after a pressurization process of pressurizing a cuff pressure indicating an inner pressure of the cuff to a pressure greater than a specified pressure, measure the blood pressure of the user based on a pulse wave signal in a depressurization process of depressurizing the cuff pressure;
a determination unit configured to determine whether or not an arrhythmia occurs in the user based on the pulse wave signal in the depressurization process; and
a mode setting unit configured to set one of a first mode for executing the determining of the arrhythmia and a second mode for not executing the determining of the arrhythmia, wherein
the blood pressure measurement unit,
sets a first depressurization speed of the cuff pressure when the first mode is set to be lower than a second depressurization speed of the cuff pressure when the second mode is set; and
sets the first depressurization speed to be lower the longer a time until the cuff pressure reaches a predetermined pressure in the pressurization process or sets the first depressurization speed to be lower the larger a size of the cuff estimated based on the time.

4. The sphygmomanometer according to claim 1, wherein
the blood pressure measurement unit
estimates a size of the cuff based on a time until the cuff pressure reaches a predetermined pressure in the pressurization process, and
sets the first depressurization speed to be lower the larger the estimated size of the cuff.

5. A sphygmomanometer configured to measure a blood pressure by compressing a part of a user to be measured with a cuff, the sphygmomanometer comprising:
a blood pressure measurement unit configured to, after a pressurization process of pressurizing a cuff pressure indicating an inner pressure of the cuff to a pressure greater than a specified pressure, measure the blood pressure of the user based on a pulse wave signal in a depressurization process of depressurizing the cuff pressure;
a determination unit configured to determine whether or not an arrhythmia occurs in the user based on the pulse wave signal in the depressurization process; and
a mode setting unit configured to set one of a first mode for executing the determining of the arrhythmia and a second mode for not executing the determining of the arrhythmia, wherein
the blood pressure measurement unit sets a first depressurization speed of the cuff pressure when the first mode is set to be lower than a second depressurization speed of the cuff pressure when the second mode is set;
a winding strength detection unit configured to detect a winding strength of the cuff with respect to the part of the user to be measured based on the cuff pressure in the pressurization process and a capacity change of the cuff is further provided, and
the blood pressure measurement unit sets the first depressurization speed to be lower the smaller the winding strength.

6. The sphygmomanometer according to claim 1, wherein
the blood pressure measurement unit sets the second depressurization speed based on an estimated pulse pressure that is a difference between an estimated systolic blood pressure and an estimated diastolic blood pressure, and a predetermined pulse rate necessary for measuring the blood pressure of the user.

7. A method for measuring a blood pressure by a sphygmomanometer configured to measure a blood pressure by compressing a part of a user to be measured with a cuff, the method comprising:
measuring, after a pressurization process of pressurizing a cuff pressure indicating an inner pressure of the cuff to a pressure greater than a specified pressure, the blood pressure of the user based on a pulse wave signal in a depressurization process of depressurizing the cuff pressure;
determining whether or not an arrhythmia occurs in the user based on the pulse wave signal in the depressurization process; and
setting one of a first mode for executing the determining of the arrhythmia and a second mode for not executing the determining of the arrhythmia, wherein
a first depressurization speed of the cuff pressure when the first mode is set is lower than a second depressurization speed of the cuff pressure when the second mode is set; and
the method further includes, setting the first depressurization speed to be lower the smaller a pulse wave amplitude indicating an amplitude of the pulse wave signal in the pressurization process, or setting the first depressurization speed to be lower the smaller the pulse wave amplitude indicating the amplitude of the pulse wave signal in a predetermined period in the past.

8. A method for measuring a blood pressure by a sphygmomanometer configured to measure a blood pressure by compressing a part of a user to be measured with a cuff, the method comprising:
measuring, after a pressurization process of pressurizing a cuff pressure indicating an inner pressure of the cuff to a pressure greater than a specified pressure, the blood pressure of the user based on a pulse wave signal in a depressurization process of depressurizing the cuff pressure;
determining whether or not an arrhythmia occurs in the user based on the pulse wave signal in the depressurization process; and
setting one of a first mode for executing the determining of the arrhythmia and a second mode for not executing the determining of the arrhythmia, wherein
a first depressurization speed of the cuff pressure when the first mode is set is lower than a second depressurization speed of the cuff pressure when the second mode is set; and
the method further includes, setting the first depressurization speed to be lower the longer a time until the cuff pressure reaches a predetermined pressure in the pressurization process, or setting the first depressurization speed to be lower the larger a size of the cuff estimated based on the time.

9. A method for measuring a blood pressure by a sphygmomanometer configured to measure a blood pressure by compressing a part of a user to be measured with a cuff, the method comprising:
measuring, after a pressurization process of pressurizing a cuff pressure indicating an inner pressure of the cuff to a pressure greater than a specified pressure, the blood pressure of the user based on a pulse wave signal in a depressurization process of depressurizing the cuff pressure;
determining whether or not an arrhythmia occurs in the user based on the pulse wave signal in the depressurization process; and
setting one of a first mode for executing the determining of the arrhythmia and a second mode for not executing the determining of the arrhythmia, wherein
a first depressurization speed of the cuff pressure when the first mode is set is lower than a second depressurization speed of the cuff pressure when the second mode is set; and
the method further includes,
detecting a winding strength of the cuff with respect to the part of the user to be measured based on the cuff pressure in the pressurization process and a capacity change of the cuff, and
setting the first depressurization speed to be lower the smaller the winding strength.
